(19) United States
(12) Patent Application Publication
(10) Pub. No.: US 2024/0366843 A1
(43) Pub. Date: Nov. 7, 2024

(54) POLYSACCHARIDE COATING

(71) Applicant: Alexander RUEBBEN, Monaco (MC)

(72) Inventor: Alexander RUEBBEN, Monaco (MC)

(21) Appl. No.: 18/774,991

(22) Filed: Jul. 17, 2024

Related U.S. Application Data
(63) Continuation of application No. 16/962,250, filed on Jul. 15, 2020, now abandoned.

(30) Foreign Application Priority Data
Jan. 15, 2018 (DE) 10 2018 100 748.5

(51) Int. Cl. A61L 29/08; A61L 29/16; C08L 5/02

(52) U.S. Cl. CPC A61L 29/085; A61L 29/16; C08L 5/02; A61L 2300/222; A61L 2300/252; A61L 2300/412; A61L 2300/416; A61L 2300/428; A61L 2300/43; A61L 2420/02

(57) ABSTRACT

The invention relates to a method for coating the balloon of a balloon catheter, wherein the surface of the balloon is wetted at least partially with a first solution containing a polysaccharide and the part of the surface of the balloon wetted with the first solution is wetted with a second solution containing an active agent. In this way, the balloon is provided with an active agent layer which is effectively applied to the inner wall of the vessel when the balloon is inflated and moreover results in a delayed long-lasting release of the active agent.

### RELATED APPLICATIONS

This application is a Continuation of U.S. patent application Ser. No. 16/962,250 filed on Jul. 15, 2020, which is a National Phase of PCT Patent Application No. PCT/EP2018/080963 having International filing date of Nov. 12, 2018, which claims the benefit of priority of German Patent Application No. 10 2018 100 748.5 filed on Jan. 15, 2018. The contents of the above applications are all incorporated by reference as if fully set forth herein in their entirety.

### FIELD AND BACKGROUND OF THE INVENTION

The invention relates to a method for coating the balloon of a balloon catheter, wherein the surface of the balloon is at least partially wetted with a first solution containing a polysaccharide. Furthermore, the invention relates to the balloon and the balloon catheter themselves.

In medicine so-called “minimally invasive procedures” play an ever-increasing role. For the treatment of vasoconstrictions such as arteriosclerosis percutaneous transluminal angioplasty (PTA) by means of balloon dilatation is frequently employed. For this purpose, a balloon catheter provided in its distal area with a balloon inflatable by the infeed of a fluid is brought to the stenotic site (vasoconstriction) with the help of a guide catheter. At the stenotic site the balloon is inflated causing deposits/plaque inhibiting the flow of blood to be pressed against or into the vessel wall so that unhindered perfusion is restored. After successful treatment and subsequent folding up/collapsing of the balloon, the balloon catheter is withdrawn from the vascular system and removed.

However, after an initially successful angioplasty, a new constriction may in some cases occur in the treated vessel area. Such restenosis is usually due to cell proliferation in the corresponding area of the vessel, i.e. cells of the blood vessel grow into the vessel lumen and cause the blood flow to be obstructed again. To prevent this, balloon catheters coated with medical substances are increasingly put to use. Such medical substances usually have an inhibitory effect on proliferation, especially on the smooth muscle cells (SMC), and in this way should prevent restenosis caused by excessive growth of these cells. The medical substance is located on the outside of the balloon and is transferred from the balloon to or into the inner wall of the vessel during balloon dilatation.

Typically, the balloon of the balloon catheter is coated by applying an active ingredient dissolved in a solvent to the surface of the balloon, with the solvent evaporating after application of the solution. The active agent will then be present in the form of a layer on the surface and can be applied while balloon dilatation takes place. With this coating method, especially the adherence of the active agent on the balloon has turned out to be problematic.

For example, in publications U.S. Pat. Nos. 5,102,402 and 6,129,705 information has been put forward as to how the adherence of an active agent on the surface could be improved. Publication U.S. Pat. No. 5,102,402 describes a balloon catheter coated with medical substances. In a first variant thereof, microcapsules filled with an active agent or drug are enclosed in folds in the balloon surface and in this way are mechanically retained in their relevant positions. In a second variant the microcapsules are attached to the balloon surface with the aid of an adhesive.

Publication U.S. Pat. No. 6,129,705 contains a description of a balloon catheter the surface of which is provided with a coating into which the microcapsules filled with an active agent are completely embedded. It is to be noted, however, that filling the active agent into microcapsules and subsequently attaching the microcapsules to or embedding them in the surface of the balloon are comparatively sophisticated and thus expensive processes.

Basically, it is desirable if the surface of the balloon of the balloon catheter has been provided with a homogeneous and reproducible drug load and at the same time is characterized by a uniform and fast drug delivery to the surrounding tissue in the body. initially be treated with a first solution of the active agent and subsequently treated with a second solution of the same active agent. In this way, a more brittle, chalk-like surface is created that results in an improved release of the active agent when the balloon is pressed against the inner wall of the vessel to be treated, as compared with surface coatings produced by a treatment with a first solution only.

In the past, it has been shown to be essential for the success of the treatment that the transfer of the active ingredient occurs rapidly during the expansion of the balloon, due to the fact that the balloon can be expanded for a maximum of 30 to 60 seconds, especially in the coronary vessels. In the event this period is exceeded, ischemic conditions and even life-threatening infarcts may occur. However, known drug-coated balloons often require a longer period of time for the active substances to be sufficiently and effectively released. This leads either to the above-mentioned ischemic problems or to an insufficient drug delivery due to the shorter balloon expansion time which must be observed.

Another problem of known drug-coated balloons is that the concentration of the drugs/medical substances at the treatment site diminishes very quickly after the balloon catheter has been removed. It must therefore be ensured that the released active ingredient remains on the vessel wall for a longer time to make sure it can be gradually absorbed and is not washed away by the bloodstream.

### SUMMARY OF THE INVENTION

It is therefore the objective of the present invention to provide a method for coating the balloon of a balloon catheter or a balloon/balloon catheter, whereby the coating on the one hand enables a rapid transfer of the active substance from the balloon to the vessel wall during the expansion of the balloon and on the other hand an improved long-term effect of the active substance after transfer can be achieved.

### DESCRIPTION OF SPECIFIC EMBODIMENTS OF THE INVENTION

As proposed by the present invention, this objective is achieved by a method for coating the balloon of a balloon catheter, wherein the surface of the balloon is at least partially wetted with a first solution containing a polysaccharide and subsequently the part of the surface of the balloon wetted with the first solution is wetted with at least one second solution containing an active substance. The first solution proposed by the invention containing a polysaccharide usually does not contain an active substance, but embodiments are conceivable in which the first solution also contains an active substance.

Using the inventive balloon catheter involves its insertion into the blood vessel system and moving it forward to the treatment site, where the balloon of the balloon catheter is expanded by means of a fluid. Through the expansion, the balloon is pressed with its outer surface against the inner wall of the vessel. During this process a major portion of the balloon coating is transferred onto the inner vessel wall. After releasing the pressure and removing the balloon catheter from the vascular system, the active ingredient applied via the coating gradually penetrates into the vascular tissue, with significant concentrations of the active ingredient still being present in the treated vascular tissue even after a considerable time has elapsed after balloon dilatation.

It turned out surprisingly that similar to an adhesive the polysaccharide coating acts on the inner wall of the treated vessel causing the active ingredient to adhere much better to the vessel wall and to be less easily carried along by the blood stream, since after transfer from the balloon surface to the vessel wall it is essentially located between the vessel wall and the polysaccharide coating. The polysaccharide coating acts like a patch that lies on the vessel wall, trapping the active ingredient/agent between itself and the vessel wall and thus being capable of preventing the active ingredient from being washed away by the bloodstream over a longer period of time. Accordingly, the active substance may remain effective over a long period of time and, protected by the polysaccharide coating, is allowed to gradually enter the tissue of the vessel. It has been demonstrated that significant concentrations of the active substance could still be detected after some weeks.

Polysaccharides constitute a hydrophilic coating that undergoes a certain swelling or softening process when existing in an aqueous environment such as blood. During balloon dilatation this results in the active substance to be properly transferred onto the inner wall of the vessel. The inventive method lends itself particularly well to the application of lipophilic coatings to the balloon. In fact, it has been found that hydrophilic polysaccharides in particular are well suited to cause lipophilic active substances to be effectively transferred during balloon dilatation to the inner walls of the vessels to be treated where they enable long-lasting active agent concentrations to be achieved. In the balloons produced by the inventive method, the polysaccharide applied in the first step is advantageously at least partially covered by the active substance.

The term balloon as it is used within the scope of the present invention shall be understood to define the element of a balloon catheter that can be expanded by feeding in a fluid, irrespective of the form or material of said expandable element. Basically, balloon catheters are sufficiently known from prior art and comprise an elongated catheter probe extending from proximal to distal as well as a balloon which is arranged in the distal area. With respect to its dimensions such a catheter is suitably designed for the insertion into a body lumen, especially into a (blood) vessel system. The relevant dimensions of such catheters may vary depending on whether the blood vessel is, for example, a coronary artery, an intracranial blood vessel or an artery in the lower leg. Moreover, the balloon catheter is provided with means for supplying a fluid to the balloon. This may be a supply lumen extending over the length of the balloon catheter.

In addition, the inventive balloon catheter may not only be used for the local administration of active substances but also for the placement of a stent (endoprosthesis) in a body lumen. Stents are tube-like supporting structures implanted into a body lumen, for example a blood vessel, with a view to keeping it permanently open. Stents of this nature may be of self-expanding design or expanded with the help of a balloon. For this purpose, the stent is crimped onto the balloon and introduced into the body lumen with the aid of a balloon catheter. At the desired placement site, the balloon is inflated by feeding in a fluid, which also causes the stent to expand and thus be anchored in the body lumen. Using the inventive balloon enables the relevant active substance to be applied at the same time to the wall of the body lumen. Finally, the balloon is deflated and removed from the body lumen whereas the stent remains in the body lumen.

In the event of a further embodiment of the invention, the layer of active substance applied with the second solution can be wetted with another liquid containing water and/or at least one alcohol. The active ingredient layer produced by the treatment with the second (active ingredient containing) solution will be attacked by the water and/or at least one alcohol containing liquid thus making the surface more porous or causing it to become brittle partially. The entire coating becomes more brittle and less transparent visually, that is it looks milkier. The surface so produced has a chalk-like, and under certain circumstances also non-crystalline consistency, which allows a higher removal efficiency of the active ingredient than in the case of a coating created only by wetting the first layer with the second solution of the active ingredient.

The water and/or at least one alcohol containing liquid is, in particular, an aqueous solution containing an alcohol and/or ketone. The concentration of the alcohol and/or ketone in the aqueous solution typically ranges between 10 and 70% (v/v), preferably between 30 and 65% (v/v), further preferred between 50 and 60% (v/v), and especially preferred is approx. 55% (v/v). Basically suitable are alcohols and ketones that can be mixed with water, wherein a blend consisting of several alcohols and/or ketones may also be employed, in which case the above-named preferred concentration details shall apply as a whole. Preferred is the use of ethanol, methanol, acetone and/or isopropanol, with ethanol being mostly preferred. Moreover, the aqueous solution may comprise an azeotropic solvent blend, in particular an alcohol/water mixture, with an ethanol/water mixture being preferred. With a view to increasing the amount of active substances present on the balloon, it would also be conceivable to provide the water and/or at least one alcohol containing liquid with an additional amount of active agent.

In the method proposed by the invention, the surface of the balloon is at least partially wetted with a first solution containing a polysaccharide. In addition to the dissolved polysaccharide, this first solution may contain other substances, but it may also be a pure polysaccharide solution. Subsequently, the part of the surface of the balloon wetted with the first solution is wetted with a second solution containing an active agent, with the second solution preferably containing no polysaccharide.

By wetting the surface of the balloon with the first solution with a polysaccharide, a layer is created on the surface which serves as the basis for a homogeneous and reproducible active ingredient loading and which enables a rapid transfer of the active ingredient during balloon expansion as well as a prolonged absorption of the active ingredient after treatment.

During wetting, especially in the event methylene chloride is used, the balloon material can absorb solvents which are released again within a few hours under normal conditions.

According to a particularly advantageous embodiment, the surface of the balloon is at least partially wetted with a primary solution containing an active substance before the surface of the balloon is at least partially wetted with a first solution containing a polysaccharide. This results in the sequence of coating the balloon surface with a primary solution containing the active substance, then the first solution containing the polysaccharide and finally the second solution containing the active substance. Usually, the active substance contained in the primary solution and the active substance contained in the second solution are identical. All statements regarding the composition of the second solution apply in the same way to the primary solution. However, the concentrations of the active substance may differ between the second solution and the primary solution; for example, the second solution may possibly contain a higher concentration of active substance than the primary solution, so that the major amount of the active substance is applied to the balloon via the second solution.

It has surprisingly been found that when the primary solution containing the active ingredient is applied to the surface of the balloon, islands or pockets of active ingredient had formed, i.e. local accumulations of the active ingredient. These result in an improved adhesion of the polysaccharide, which in turn leads to further improvements of the effects described hereinbefore. To a certain extent, a comparable effect can be achieved if the first solution, in addition to the polysaccharide, also contains active substance. The islands of active ingredients that are thus formed will improve the adhesion of the polysaccharide.

The polysaccharide in the first solution preferably exists in an alcohol containing solution. Aside from one or several alcohols this solution may in particular also contain water. An aqueous-alcoholic solution is advantageous in that it dissolves the polysaccharide well on the one hand, while the organic portion in the solution ensures rapid drying after wetting. The concentration of the alcohol or alcohols in the first solution typically ranges between 10 and 70% (v/v), preferably between 30 and 65% (v/v), further preferred between 50 and 60% (v/v), and especially preferred is approx. 55% (v/v). Suitable alcohols are those that are capable of dissolving polysaccharide. As a rule, such alcohols can also be mixed with water. Preferred are ethanol, methanol, and isopropanol, with ethanol being especially preferred.

Expediently, the average molar mass of the polysaccharide ranges between 10,000 and 100,000,000 Da. A mean molar mass of between 20,000 and 80,000 Da has proved to be especially suitable for the purpose. Branched polysaccharides are preferred. Furthermore, it is advantageous if the polysaccharide has long polysaccharide chains. Moreover, the polysaccharide should be water soluble. The content of polysaccharides of the first solution preferably amounts to between 1 and 15% (w/w), further preferred between 2 and 10% (w/w), and especially preferred between 3 and 8% (w/w).

Preferably, the polysaccharide is a branched polysaccharide. Also suitable are mixtures composed of several polysaccharides and modified polysaccharides. Preferred are dextrans, in particular natural dextrans. Dextrans are high-molecular, branched polymers composed of glucose units. They are produced, inter alia, by bacteria of the genus Leuconostoc. They are used as blood plasma substitutes or as carriers in chromatography.

In particular, the dextran may be a natural dextran, with dextran 40 having an average molar mass of approx. 40,000 Da being especially preferred.

However, aside from dextrans other polysaccharides may basically be employed as well. An example of a modified polysaccharide that can be used is hydroxyethyl starch (HES).

In principle, either the entire balloon surface or only a part of the balloon surface, e.g. the area of the surface coming into contact with the fabric during expansion, can be coated by means of the method proposed by the invention. The balloon may, in particular, consist of a cylinder-shaped area and at least one tapering/conical area. For example, only the cylinder-shaped portion of the balloon may in this case be coated with an active substance in accordance with the invention or the cylinder-shaped portion and a conical portion.

The second solution may be saturated with respect to the active substance. The solvent for the second solution as well as the primary solution may be, for example, methylene chloride, chloroform, alcohol, especially ethanol, methanol or isopropanol, acetone, diethyl ether, liquid hydrocarbons such as pentane, hexane, heptane, cyclohexane or octane, toluol, tetrahydrofuran (THF) or ethyl acetate. Furthermore, solvent mixtures or blends may also be employed. Preferably, it may be a dissolution of the active substance in methylene chloride or chloroform.

All the steps for wetting the surface of the balloon with a liquid can be carried out by immersing the balloon in the respective solution. As a rule, the balloon is immersed for a maximum period of 1 min., typically for 10 to 30 s, with the balloon being in at least partially expanded state during immersion. The immersed balloon should then be pulled out of the first solution at a maximum speed of 10 mm/s. Even more favorable would be to withdraw the balloon at a speed of less than 5 mm/s, preferably at a speed ranging between 0.5 mm/s and 2 mm/s. Withdrawing the balloon slowly enables the surface to dry gradually and slowly.

As an alternative to wetting the balloon by immersion other methods may also be adopted, for example spraying.

In addition, before being wetted with the first solution the surface of the balloon may be cleaned and/or provided with structuring or given a profiled contour. The balloon surface may, for example, be structured or profiled mechanically, thermally or chemically. Such structuring or profiled contour may be brought about in particular by roughening the surface. Advantageously, depressions having a depth of between 5 and 50 μm and a diameter ranging between 5 and 50 μm are thus created on the surface by such an enlargement of the balloon surface. The profiled contour improves the adhesion of the polysaccharide solution.

Furthermore, having been wetted with the first solution containing a polysaccharide and prior to and/or after wetting with the second solution containing an active substance the surface of the balloon can be wetted with an additional solution of the same or another active substance. In this manner, the active agent load will be increased. Wetting the surface with said additional solution may also bring about an at least partial embrittlement of the entire coating. Moreover, the entire coating may appear less transparent visually and thus have a milkier look. Overall, wetting with the additional solution of the active ingredient causes a higher active ingredient load to be released.

Basically, the balloon may be wetted with as many additional solutions as desired of which not all need contain the active agent. If thought expedient, an additional solution may also contain another type of active agent.

For example, said additional solution may contain the active agent dissolved in methylene chloride. Preferably, the concentration of the solution should be lower than that of the first solution, for example 100 mg/ml. Other solvents such as for example chloroform or ethanol or solvent mixtures may be employed as well. The surface of the balloon can be wetted by immersing the balloon into the additional solution while other techniques, for example spraying, may also be applied, however.

Furthermore, the surface of the balloon may also be dried after wetting with the primary solution, wetting with the first solution containing a polysaccharide, wetting with the second solution containing an active substance and wetting with the water and/or at least one alcohol containing liquid. For example, the balloon may have a longitudinal axis and be rotated around its longitudinal axis during the drying process. To achieve an as uniform as possible drying effect the longitudinal axis of the balloon can be positioned horizontally immediately after wetting has been completed. The balloon can then be rotated in a stream of air around its longitudinal axis.

The active agent used is, in particular, a drug or medical substance that has a proliferation-inhibiting effect preventing a vasoconstrictive overgrowing of the vessel site previously expanded by the balloon. The invention is particularly suitable in the case of hydrophobic agents. The active agent may in particular be selected from the following: Tretinoin, orphan receptor agonists, elafin derivatives, corticosteroids, steroid hormones, paclitaxel, rapamycin, tacrolimus, hydrophobic proteins as well as substances modifying cell proliferation. Mixtures of these active agents may also be used. Moreover, derivatives of the above cited active agents may also be of use, wherein said derivatives may in particular be salts, esters, and amides. As steroid hormones methylprednisolone, dexamethasone or estradiol may be used, for example paclitaxel, rapamycin or tacrolimus or corresponding derivatives are particularly preferred.

Preferably, coating the balloon with the active agent takes place without solutizers being used. Solutizers for example are: Phosphatidylcholine, polyethoxylated castor oil, cardiolipin, cholesterol as well as mixtures thereof.

Within the scope of the invention, the balloon of a balloon catheter has a surface on which at least partially a coating is arranged consisting of a first layer with a polysaccharide being directly attached on the surface. Arranged on this first layer there is a second layer containing an active substance. The coating is preferably homogeneous over the entire area covered. By wetting the active substance layer with a further liquid containing water and/or at least one alcohol, this active substance layer becomes brittle. In particular, the surface can thus have a chalk-like, possibly also non-crystalline structure. Furthermore, the surface of the balloon may be coated either completely or only in part. The balloon may, in particular, comprise of a cylinder-shaped area and at least one tapering/conical area. In this case, for example, only the cylinder-shaped part of the balloon or the cylinder-shaped portion and a conical area may be coated as proposed by the invention. The inventive balloon can be manufactured by adopting the inventive method. It ensures rapid, homogeneous and high-volume drug delivery to the surrounding tissue in the body, which adheres to the vessel wall for a long time due to the applied layer of a polysaccharide.

The balloon catheter proposed by the present invention comprises the above described inventive balloon and offers the same advantages as the inventive balloon. The balloon catheter of the invention is typically provided with lumens, preferably at least two lumens, one lumen serving for the supply of fluid and for pressurization and providing connection to the interior of the balloon, whereas the other lumen serves for receiving a guidewire which is first advanced to the target site in the blood vessel in order to subsequently bring the balloon catheter to the target site via the guidewire. In this context, two different state-of-the-art systems are essentially known, namely Over-The-Wire (OTW) and Rapid Exchange (Rx) balloon catheters. The balloon catheter provided by the invention can be either an OTW or an Rx balloon catheter. While in an OTW catheter the lumen for the guidewire extends over the entire length of the catheter from proximal to distal, the Rx catheter has a separate feed opening for the guidewire (Rx port), where the guidewire exits the catheter at a location clearly distal to the proximal end of the catheter. Accordingly, in the case of an OTW balloon catheter the lumens that serve for the fluid supply and the guidewire run concentrically or parallel to each other from the proximal end of the catheter to the balloon, whereas in an Rx catheter this is only the case between the Rx port and the balloon. However, the section between the Rx-port and the proximal end has only one lumen for fluid supply. Typically, in areas where the catheter has two lumens, the lumens are of concentric configuration, that is, the narrower inner lumen for the guidewire extends through the wider outer lumen that serves for fluid delivery.

The proximal end of the balloon catheter is usually provided with a so-called catheter hub, i.e. a connection element for the device for fluid supply and pressurization. The connection can, for example, be a conventional Luer or Luer-Lock connection. The term proximal means towards the outside of the body, i.e. towards the attending physician, while distal means the opposite direction, i.e. towards the blood vessel to be treated. The balloon catheter is usually introduced into the human body in the groin area via the femoral artery.

Over the length of the balloon catheter radiopaque markers may be arranged at various positions, said markers serving the purpose of making the catheter visible on radiographs. In particular, said markers may be manufactured of platinum or a platinum alloy.

What is claimed is:

1. A method for coating a balloon for a balloon catheter, comprising a sequence of coating steps, said coating steps comprise:
wetting at least a part of a surface of the balloon with a primary solution containing an active substance;
wetting at least said part of said surface with a first solution containing a polysaccharide; and
wetting at least said part of said surface with at least one second solution containing said active substance.

2. The method of claim 1, further comprising, subsequent to said wetting with said second solution, wetting at least said part of said surface with a liquid, said liquid comprises water and/or at least one alcohol and/or at least one ketone.

3. The method of claim 2, wherein a concentration of said at least one alcohol and/or said at least one ketone in said liquid ranges 10-70% (v/v).

4. The method of claim 1, further comprising, subsequent to said wetting with said second solution, wetting at least said part of said surface with an additional solution that comprises said active substance and/or another active substance.

5. The method of claim 4, further comprising, subsequent to said wetting with said additional solution, wetting at least said part of said surface with a liquid, said liquid comprises water and/or at least one alcohol and/or at least one ketone.

6. The method of claim 5, wherein a concentration of said at least one alcohol and/or said at least one ketone in said liquid ranges 10-70% (v/v).

7. The method of claim 5, wherein said liquid comprises ethanol, methanol, acetone and/or isopropanol.

8. The method of claim 1, wherein each of said first solution and/or said second solution independently comprises at least one alcohol.

9. The method of claim 8, wherein each of said first solution and/or said second solution independently comprises water and a concentration of said at least one alcohol ranges 10-70% (v/v).

10. The method of claim 8, wherein at least one alcohol is selected from the group consisting of ethanol, methanol, and/or isopropanol.

11. The method of claim 9, wherein a concentration of said alcohol ranges 30-65% (v/v).

12. The method of claim 1, wherein an average molar mass of the polysaccharide amounts to between 10,000 and 100,000,000 Da.

13. The method of claim 1, wherein said polysaccharide is water-soluble.

14. The method of claim 1, wherein said polysaccharide is a dextran.

15. The method of claim 1, wherein said active substance is hydrophobic.

16. The method of claim 15, wherein said active substance is selected from the group consisting of Tretinoin, orphan receptor agonists, elafin derivatives, corticosteroids, steroid hormones, paclitaxel, rapamycin, tacrolimus, hydrophobic proteins and/or substances modifying cell proliferation.

17. The method of claim 1, wherein said first solution is devoid of said active substance.

18. A balloon for a balloon catheter, characterized by a surface having a coating on at least a part of said surface, said coating is obtained by the method of claim 1.

19. The balloon of claim 18, wherein said active substance covers at least part of said polysaccharide.

20. A balloon catheter comprising the balloon of claim 18.
